(19) United States
(12) Patent Application Publication
(10) Pub. No.: US 2014/0364390 A1
(43) Pub. Date: Dec. 11, 2014

(54) XYLOGLUCAN-BASED COMPOSITIONS FOR THE TREATMENT OF GASTROINTESTINAL DISORDERS

(71) Applicant: Novintethical Pharma, Sagl, Lugano (CH)

(72) Inventors: Marco Di Fulvio, Soriano nel Cimino (IT); Miguel Angel Alonso, Barcelona (ES); Michele Giuseppe Di Schiena, Robecco sul Naviglio (IT)

(73) Assignee: Novintethical Pharma, Sagl, Lugano (CH)

(21) Appl. No.: 14/092,365

(22) Filed: Nov. 27, 2013

(30) Foreign Application Priority Data
Jun. 11, 2013 (IT) MI2013A000960

(51) Int. Cl. A61K 31/716; A61K 45/06

(52) U.S. Cl. CPC A61K 31/716; A61K 45/06
USPC 514/54; 536/123.12

(57) ABSTRACT

Described herein are compositions including xyloglucans as an active ingredient useful in the treatment of gastro-intestinal disorders or of disorders originating from the gastro-intestinal system and transferred to other systems, such as the genitourinary system. The compositions may have xyloglucans or an extract containing xyloglucans, and may further include other active ingredients.

### FIELD OF THE INVENTION

The invention refers to compositions comprising xyloglucans and the use thereof as active ingredients in compositions for the treatment of gastro-intestinal disorders, in particular forms of diarrhoea of various origin.

### BACKGROUND

Diarrhoea is an often invalidating and dangerous symptom, especially in children and the elderly, of many gastro-intestinal diseases. Acute diarrhoea is mainly caused by intestinal infections, but it may also be due to use of drugs, radiotherapy treatments and other pathological conditions (diverticulitis, heavy metal intoxications, intestinal ischemia, allergies or intolerances).

Acute infectious diarrhoea represents a serious problem in developing countries given that it is deemed to annually cause the death of at least 4 million children below 5 years of age.

Chronic diarrhoea is mainly due to the irritable bowel syndrome or coeliac disease or intestinal inflammatory diseases (Crohn's disease, ulcerative rectocolitis).

In the light of the various etiologies, there are available various therapeutic options based on the administration of antibiotics/antibacterial agents, antispasmodic/anticholinergic agents, probiotics, opioid receptor agonists. However, some of these treatments should be administered very cautiously, given that they do not intervene at the level of the causal pathologic process.

Thus there arises the need for further therapeutic treatments capable of replacing or being administered alongside the ones available currently.

### SUMMARY OF THE INVENTION

Now, it has been surprisingly discovered that xyloglucans have a filmogenic effect at the intestinal mucosa level capable of reducing the permeability of the narrow junctions of the intestinal mucosa and thus hindering the entry of the pathogens causing the acute intestinal infections. The filmogenic effect is not influenced by the variation of the pH.

Thus, the invention provides pharmaceutical compositions comprising, as active ingredients, xyloglucans or extracts containing them combined with suitable excipients and possibly with other active ingredients useful for the treatment of gastro-intestinal and genitourinary diseases.

### DETAILED DESCRIPTION

Xyloglucans are molecules constituted by a linear skeleton of β-1,4-glucans with short lateral ramifications. The latter bind due to xylose bound to oxygen in position 6 of sugar. Such lateral chains may also contain other sugars such as arabinose and fucose.

Xyloglucans belong to the family of hemicelluloses which associate with cellulose within the cell walls of the upper plants. A particularly rich source of xyloglucan is the seed of the tamarind (Tamarindus indica), a tropical tree from East Africa.

Extracts of tamarind seeds rich in xyloglucans are known and they were used in the medical field mainly as viscosifying agents in ophthalmic compositions (U.S. Pat. No. 6,056,950), as muco-adhesive agents (WO2006131262), lacrimal substitutes (WO2009/044423), as anti-infection agents (WO2011147767) and as anti-inflammatory agents (WO2011147768).

Xyloglucans extracts from Tamarindus indica are available in the market from example from Indena (Italy) (Xilogel®) and from DSP Gokyo Food & Chemical (Japan) (Glyloid®).

Examples of suitable forms of administration comprise oral forms such as capsules, tablets, solutions, suspensions, granules, gels, and the like.

Examples of other active ingredients with which xyloglucans can be combined comprise antibiotics, antimotility agents, steroidal or non-steroidal anti-inflammatories, compounds for the treatment of gastrointestinal meteorism (simethicone and the like), mesalazine, sucralfate, natural and synthetic polysaccharides such as for example pectines, chitosan (animal or vegetable), hyaluronic acid, Guar gum, xanthan gum, animal gelatins, vegetable proteins such as for example the pea protein, cellulose and hemicellulose and derivatives such as for example hydroxypropyl cellulose, carragenines, carbomers, cross-linking/polymerising compounds such as ferulic acid; polyphenols, such as for example gall polyphenols, grape seed polyphenols, probiotics, such as for example Lactobacilli, Bifidobacteria, yeasts and the like.

A preferred combination is that with gelatin tannate which has been available in the market for many years as a medical device for the treatment of acute diarrhoea.

In the compositions of the invention, xyloglucans may be present in a wide range of concentration which depends on the type of composition and therapeutic indication they are intended for.

The range of concentration of xyloglucan referred to the single administration dose is comprised between 0.5 mg/dose and 2000 mg/dose, preferably between 1 mg/dose and 500 mg/dose.

The compositions of the invention can be used for the treatment and the prevention of gastro-intestinal disorders and of disorders originating in the gastro-intestinal system and transferred to other systems, such as for example the genitourinary system. Actually, it is known that the gram negative bacteria, in particular Escherichia coli, present in the intestine may proliferate in this organ and migrate into the urinary system where they cause 90% of the genitourinary infections such as cystitis, cysto-pyelitis and the like.

In particular, the compositions of the invention can be used for the prevention of the proliferation of pathogens in the gastro-intestinal system and the transfer thereof to other systems of the human organism through the narrow intestinal junctions, as well as for the protection of the intestinal mucosa against chemical or physical agents that may reduce the functionality and natural regeneration of the intestinal epithelium and for the reduction of the para-cellular flow of pathogens through the intestinal walls.

The compositions of the invention also revealed to be useful for the prevention and treatment of damages of the intestinal mucosa and the consequent inflammatory conditions such as diverticulosis and of the early stages of diverticulitis; for the treatment of the symptoms consequent to alimentary allergies (for example lactose intolerance, gluten intolerance etc.); for the prevention and the treatment of digestion disorders (production of gas, meteorism, stomach rumble, flatulence); for the prevention and the treatment of damages of the intestinal mucosa deriving from local inflammatory conditions, both of temporary and chronic origin, in particular for the treatment of Crohn's disease, ulcerative colitis or Irritable Bowel Syndrome (IBS).

The compositions of the invention may be advantageously used for the treatment of diarrhoea in combination with electrolytes for oral rehydration, as a mucomimetic or for hindering the adhesiveness of the bacteria to the mucosa and the subsequent proliferation which leads to dysbacteriosis, possibly combined with probiotics or tyndallised bacteria.

The compositions of the invention efficiently protect the mucosa and reduce the adhesion of some pathogens, for example gas producing bacteria, thereto.

The following examples illustrate the invention further in detail.

### Example 1

Composition for the prevention and the treatment of diarrhoea; 3 g single dose sachet

| | | |
|---|---|---|
| Xyloglucan | 0.100 | g |
| Inulin | 1.650 | g |
| Maltodextrin | 1.195 | g |
| Stevioside (Stevia) | 0.015 | g |
| Tutti frutti flavour (Firmenich) | 0.015 | g |
| Colouring agent E160 (a) (Betacarotene) | 0.025 | g |

### Example 2

Composition for the prevention and the treatment of diarrhoea; 0.200 g rigid capsule

| | | |
|---|---|---|
| Xyloglucan | 0.004 | g |
| Matricaria d.e. | 0.026 | g |
| Pectin | 0.050 | g |
| Dimethicone | 0.020 | g |
| Kaolin | 0.020 | g |
| Magnesium stearate | 0.080 | g |

### Example 3

Composition for the prevention and treatment of diarrhoea, 0.100 g tablet

| | | |
|---|---|---|
| Xyloglucan | 0.002 | g |
| Lactose | 0.063 | g |
| Anhydrous colloidal silica | 0.002 | g |
| Micro-crystalline cellulose | 0.030 | g |
| Magnesium stearate | 0.003 | g |

### Example 4

### Biological Tests

E. coli invasion method was used using the CacoGoblet® (Admecell, Alameda, Calif., USA) commercial kit. According to a first protocol, the Caco-2 cells were pre-treated using xyloglucan at the concentration of 5 mg/ml for 1, 4 or 24 hours. The cells were thus inoculated for 30 minutes using E. coli (1.26×10⁸ CFU/well). Then, there followed the determination of the para-cellular flow and the permeability by measuring the passage of the Lucifer Yellow colouring agent after each exposure to the treatment using xyloglucan at 1, 4 and 24 hrs. This assay allowed evaluating the integrity of the cell junctions in the presence of the substance under scrutiny.

The transepithelial electrical resistance (TEER) which provides a direct measurement of the barrier function and which is a further parameter of the wholeness of barrier at the narrow junction level was also evaluated.

The same measurements of passage Lucifer Yellow colouring agent and the TEER were also carried out using a treatment protocol, in which the Caco-2 cells were first inoculated using E. coli according to the previously described methods and thus treated after 1, 4 and 24 hours using xyloglucan.

Upon testing the passage of Lucifer Yellow colouring agent in the prevention protocol, the treatment using xyloglucan caused a marked reduction of the para-cellular flow at all considered times, from 8.79% of the initial value after challenge using E. coli to 3.84, 3.45 and 3.95% at 1, 4 and 24 hrs, respectively. Xyloglucan also revealed to be capable of reducing the adhesion of E. coli on the intestinal mucosa.

In the treatment protocol, xyloglucan was capable of reducing the para-cellular flow from 8.11 to 3.44 after 1 hr and from 8.28% to 3.01% after 4 hrs. The recovery percentage of the wholeness of the barrier, measured by the TEER values, increased from 0 to 28% after one hour rising up to 81% after 4 hours. The wholeness of barrier could still be observed after 24 hours.

The results clearly reveal the capacity of xyloglucan to act as a filmogenic agent capable of restoring the functionality of barrier and reducing permeability both in the post-infection treatment and in the prevention model. Xyloglucan is thus efficient at protecting the intestinal mucosa from adhesion and damage caused by E. coli and protecting the structure of the narrow cell junctions with long-term efficiency.

We claim:

1. A composition for use in the treatment of gastro-intestinal disorders or of disorders originating in the gastro-intestinal system and transferred to other systems, said composition comprising xyloglucans or extracts containing xyloglucans.

2. A composition according to claim 1, wherein the disorder originates in the gastro-intestinal system and is transferred to the genitourinary system.

3. A composition according to claim 1, wherein the gastro-intestinal disorder is diarrhoea.

4. A composition according to claim 1, wherein said treatment is selected from the group consisting of (a) the treatment of damages of the intestinal mucosa and of the consequent inflammatory conditions selected from diverticulosis or early stages of diverticulitis; (b) the treatment of symptoms consequent to alimentary allergies; (c) the prevention and treatment of digestive disorders; and (d) the prevention and treatment of damages of the intestinal mucosa deriving from local inflammatory conditions, of temporary or chronic origin.

5. A composition according to claim 4, wherein said composition is used for the treatment of a disorder selected from Crohn's disease, ulcerative colitis, Irritable Bowel Syndrome (IBS), diverticolosis, early stages of diverticulitis, gluten or lactose intolerance, stomach rumble, meteorism, or flatulence.

6. A composition according to claim 1, wherein said composition further comprises one or more active ingredients and at least one pharmaceutically acceptable excipient.

7. A composition according to claim 6, wherein the one or more active ingredients are selected from the group consisting of antibiotics, antimotility agents, steroidal or non-steroidal anti-inflammatories, compounds for the treatment of gastrointestinal meteorism, mesalazine, and sucralfate.

8. A composition according to claim 6, wherein the at least one pharmaceutically acceptable excipient is selected from the group consisting of natural or synthetic polysaccharides such as pectins, chitosan (animal or vegetable), hyaluronic acid, guar gum, xanthan gum, animal gelatins, vegetable proteins, cellulose and hemicellulose, hydroxypropyl cellulose, carrageenans, carbomers, ferulic acid; polyphenols, probiotics, gelatin tannate, and electrolytes.

9. A method of treatment of gastro-intestinal disorders or of disorders originating in the gastro-intestinal system and transferred to other systems, said method comprising the steps of:
(a) providing a composition comprising xyloglucans or extracts containing xyloglucans
(b) administering the composition to a patient in need thereof, said patient having a gastro-intestinal disorder or a disorder originating in the gastro-intestinal system and transferred to other systems.

10. The method of claim 9, wherein the disorder originates in the gastro-intestinal system and is transferred to the genitourinary system.

11. The method according to claim 9, where in the composition further comprises one or more active ingredients.

12. The method according to claim 11, wherein said active ingredients are selected from the group consisting of antibiotics, antimotility agents, steroidal or non-steroidal anti-inflammatories, compounds for the treatment of gastrointestinal meteorism, mesalazine, and sucralfate.

13. The method according to claim 9, wherein said disorder is diarrhoea.
